(19) United States
(12) Patent Application Publication
(10) Pub. No.: US 2026/0215495 A1
(43) Pub. Date: Jul. 30, 2026

(54) Ionic Personal Exhalant Filter

(71) Applicant: Philter Labs Incorporated, Encinitas, CA (US)

(72) Inventors: John Grimm, Santee, CA (US); Andrew Lamoureux, Santee, CA (US); Gianni Grimm, Santee, CA (US)

(73) Assignee: Philter Labs Incorporated, Encinitas, CA (US)

(21) Appl. No.: 19/531,471

(22) Filed: Feb. 5, 2026

Related U.S. Application Data
(63) Continuation-in-part of application No. 18/130,544, filed on Apr. 4, 2023.
Application No. 18/130,544 is a continuation-in-part of application No. 17/885,432, filed on Aug. 10, 2022, now abandoned.
(60) Provisional application No. 63/233,175, filed on Aug. 13, 2021.
(60) Provisional application No. 63/968,951, filed on Jan. 27, 2026.

(51) Int. Cl. A24F 13/00; B03C 3/019; B03C 3/32; B03C 3/36; B03C 3/41; B03C 3/47

(52) U.S. Cl. CPC A24F 13/00; B03C 3/019; B03C 3/32; B03C 3/363; B03C 3/41; B03C 3/47

(57) ABSTRACT

An ionic personal exhalant air filter comprising, generally, a mouthpiece, a tube and high voltage electrodes configured thereto, is disclosed. Notably, the construction creates fluid dynamic properties needed to allow ionization and collection to take place. The unit is small and easily fits in a user's hand. In a preferred embodiment, the negative collector plate is realized with a ring structure having veins at an interior thereof; and the positive collector plate comprises a copper sheet rolled to a coil increasing surface area of electrodes while providing a compact unit.

### PRIORITY CLAIM

The present patent application is a Continuation-in-Part of U.S. patent application Ser. No. 18/130,544, entitled “Eco-Friendly Personal Exhalant Filter,” filed on Apr. 4, 2023, which in turn, is a Continuation-in-Part of U.S. patent application Ser. No. 17/885,432 , entitled “Ionic Personal Exhalant Filter,” filed on Aug. 10, 2022 which claims benefit of the priority date of U.S. Prov. Pat. App. Ser. No. 63/233,175 filed on Aug. 13, 2021, entitled “Ionic Personal Air Filter.”

Additionally further, the present patent application claims benefit of the priority date of U.S. Prov. App. Ser. No. 63/968/951 filed on Jan. 27, 2026, entitled “Vapor Device with Ionic Exhalant Filter” Accordingly, the entire contents these U.S. provisional patent submissions are hereby expressly incorporated by reference.

### BACKGROUND OF THE INVENTION

### Field of the Invention

The present invention pertains generally to accessories for electronic smoking devices and traditional smoking in general. More specifically, the present invention relates to an ionizing exhale filter capable filtering pollutants or particulates in a user's exhale.

### Description of the Prior Art

Smoking tobacco or other plant material has notoriously been practiced in numerous cultures for many centuries. Importantly, in the last decade or so, traditional smoking of tobacco has been banned in places such as bars, casinos and beaches where smoking has traditionally been Smoking has additionally been banned on airline travel for at least about twenty years where no alternative has been adopted or provided.

Ever increasing in popularity, “electronic cigarettes/e-cigarettes” and vaporizers typically use an electrical heating element to heat a liquid usually containing a mixture of nicotine and flavorings/extract (or cannabis including THC or CBD) that produces a mist similar to smoke but without the excessive heat that causes combustion. Similarly, the use of these devices has been banned in many public places.

Also recently, medicinal and recreational use of cannabis has increased in popularity and acceptance. And accordingly, users desire to address an issue of second-hand smoke and any distinctive odor resulting from such use.

Personal hand-held filter devices have subsequently emerged wherein a user exhales smoke, vapor or mist into a device to eliminate same. An example currently on the market has been branded the Smoke Buddy®. This example uses carbon pellets to filter smoke and particles suspended in air. A still more recent product has patent protection assigned to present applicant, Philter Labs, and is entitled ‘Low Emissions Electronic Smoking Device and Emissions Filter Device, U.S. Pat. No. 10,945,458. This and similar devices utilize combinations of mesh filters, charcoal/carbon, HEPA, felt, and corrugated materials, etc. These two examples are passive filters in that there is no prime mover such as a fan; and there are not electrical connections or energized components. It has been contemplated that more active filtration may be desired and achieved in a compact device such as that introduced in the '458 patent.

More particularly, it is an object of the present invention to provide improvements to exhale and/or personal air intake filtration system(s) compact in size as compared to commercial or residential air filtrations. It is further an object of the present invention to provide an air filter system that is devoid of a prime mover such as a fan due to the personal nature and preferences of the average smoker today.

Also importantly, existing filter technology, in particular graphite filter technology, includes granular filter media which can greatly imped air flow. The Smoke Buddy® device is specifically an external filter for receiving exhaled mist; however, it's a bit bulky, awkward and irregular in shape. It is an additional object of the present invention to provide improved air flow in a smaller device that can be used to filter exhale and perhaps other means of air intake desired to be filtered.

Also known in the electro-chemical arts are ionizing air purifiers for improving air quality in office spaces and private residences, etc. These relatively large units (about 18″ tall, or more) use a high voltage electrical potential to strip electrons from particles suspended in air resulting in a negative charge, thereto, or negative ion. Those particles are attracted to a positively charged collector plate, thereby filtering the air removing these particles. It is an additional object of present invention to utilize similar technology to filter smoke through a small, hand-held device, without fans or electrical cords employed by known plug-in room air purifiers.

### BRIEF SUMMARY OF THE INVENTION

The present invention specifically addresses and alleviates the above-mentioned deficiencies, more specifically, the present invention, in a first aspect is an ionic personal air filter filtering smoke or vapor exhale comprising: a hollow chamber portion, or cylindrical tube; a mouthpiece in fluid communication with the hollow chamber portion for receiving user exhale introduced thereto; a negative electrode configured to the hollow chamber portion (either suspended in the tube/hollow chamber portion, or at an upper portion/area thereof), the negative electrode (downstream of the mouthpiece) at the upper portion of the tube providing an electrical potential creating a plurality of ionized smoke or vapor particles; and a positive collector plate at a lower portion of the hollow chamber portion for collecting the ionized smoke particles; and a battery providing the electrical potential between the negative electrode and the positive electrode.

The invention in this first aspect is additionally characterized wherein the positive collector plate comprises a copper sheet (or foil, or mesh) rolled in a coil thereby increasing surface area in contact with the ionized smoke or vapor inside the hollow chamber portion. This first aspect further includes: a step-up high voltage generator (HVG) increasing the electrical potential between the negative electrode and the positive electrode; a one-way flow sensor senor switch to activate (or energize) the personal air filter upon exhale; wherein an air flow is sensed by an air flow sensor and the one-way flow sensor switch closes an electrical circuit connecting the battery and the negative electrode; an end cap coupled to an exit to the hollow chamber portion comprising an activated extruded carbon filter, for filtering any smoke or vapor not ionized or otherwise captured by the positive collector plate; and an outer enclosure containing the hollow chamber portion, the battery and the high voltage generator (HVG).

Importantly for desired fluid dynamic properties, the invention introduces an impact flow diverter piece for redirecting and circulating air flow configured in between the mouthpiece and hollow chamber portion, wherein the impact flow diverter piece is configured over, around, and about an exit to the mouthpiece, thereby redirecting air flow upwardly and in a reverse direction to the air flow at the exit to the mouthpiece, wherein the impact flow diverter piece further comprises apertures for subsequently redirecting airflow outwardly and downwardly passing through the hollow chamber portion.

The personal exhalant filter in this aspect is additionally characterized wherein the negative electrode comprises: a ring configured at an upper area of the hollow chamber portion; and an additional metallic structure, or veins, at an interior to the ring, the additional metallic structure providing added surface area stripping electrons from harmful compounds.

The invention is a second aspect is a personal exhalant filter for filtering smoke or vapor exhale comprising: an inner chassis having an interior, the interior housing a filter medium; a hollow chamber portion configured (extending downwardly into) to an opening in the inner chassis; a mouthpiece in fluid communication with the hollow chamber portion for receiving user exhale introduced thereto; and an outer enclosure configured at least partially over the inner chassis and at least partially encasing the hollow chamber portion.

This invention in this aspect further includes the ionizing components necessary for operation, including: an impact flow diverter piece for redirecting and circulating air flow configured at an exit of the mouthpiece and at an upper area of the hollow chamber portion; a negative electrode configured to the hollow chamber portion, either suspended in the tube or at an entrance/upper portion thereto, the negative electrode providing electrical potential creating a plurality of ionized smoke or vapor particles; and a positive collector plate at a lower portion of the tube (or hollow portion) for collecting the ionized smoke particles; a battery delivering a voltage to the personal air filter and the negative electrode; a step-up high voltage generator increasing the voltage to the negative electrode; and a one-way flow sensor senor switch to energize, or activate, the personal air filter upon exhale; wherein an air flow is sensed by an air flow sensor and the one-way flow sensor switch closes an electrical circuit connecting the battery and the negative electrode.

The invention in this second aspect is further characterized wherein the positive collector plate comprises a copper sheet rolled in a coil thereby increasing surface area in contact with the ionized smoke particles. Last but not least, the inner chassis is also a removable end cap for the invention and further comprises an activated extruded carbon filter and a HEPA (High Efficiency Particulate Air) filter, for filtering ionized smoke (or vapor) particles captured by the positive collector plate. Hence, the end cap is removable and replaceable thereby extending the useful life of the exhalant filter.

While the apparatus and method has or will be described for the sake of grammatical fluidity with functional explanations, it is to be expressly understood that the claims, unless expressly formulated under 35 USC § 112, or similar applicable law, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC § 112 are to be accorded full statutory equivalents under 35 USC § 112, or similar applicable law. The invention can be better visualized by turning now to the following drawings wherein like elements are referenced by like numerals.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is an exploded view of a personal active ion reaction exhalant air filter system of the present invention having the outer enclosure removed;

FIG. 2 is an isometric view of the outer enclosure;

FIG. 3 is a profile view of the assembled first preferred embodiment;

FIG. 4 is a cross-sectional view of the first preferred ionic exhalant filter taken from line 4-4 in FIG. 3; and

FIG. 5 is an exploded view of the sectional area taken about line 5-5 in FIG. 4.

### DETAILED DESCRIPTION OF PREFERRED EMBODIMENTS

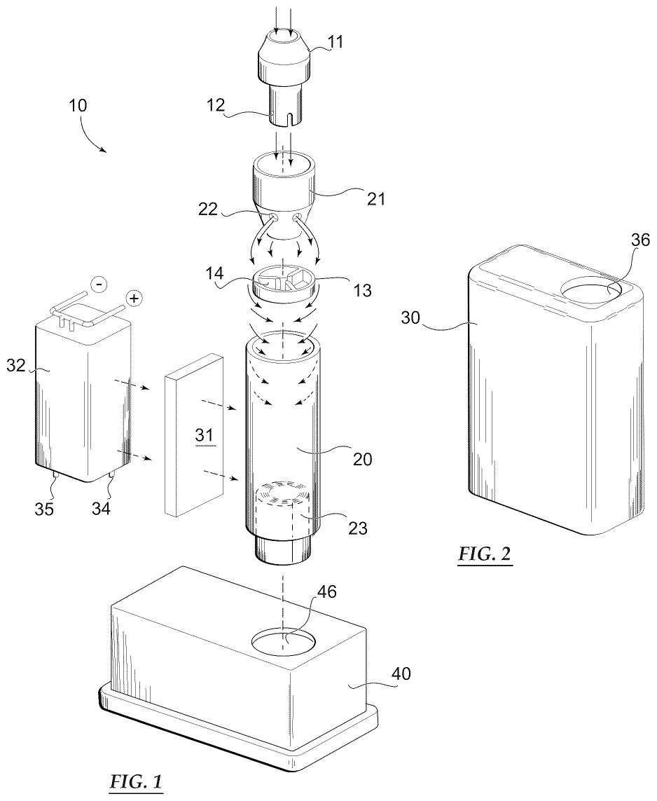

Referring initially to FIG. 1, the invention 10 is essentially a tube 20 a user is able to exhale into. The cylindrical tube 20, or more broadly, hollow exhale ionization chamber 20 portion is configured with technology that removes smoke or vapor being blown thereto at normal velocity pressure changes provided by typical adult human exhale respiration. Particularly, the smoke is able to be ionized with the novel configuration of components that can make ionization 13 and collection 23 of smoke particles possible. Such smoke or vapor particulate may contain volatile compounds, also generally referred to herein as ozone, or O³.

Now with regard to FIG. 1 through FIG. 4, the invention comprises a mouthpiece 11 coupled to the tube 20 via an impact flow diverter 21 piece. The impact flow diverter 21 is an important component required due to the desired fluid dynamic properties needed for proper smoke travel path, distance and velocity. If not for the fluid dynamics achieved, the smoke or vapor would simply fail to ionize or any ionic smoke particulate might be unable to be collected and hence pass through unfiltered. More particularly with regard to FIG. 1 and FIG. 5, the impactor flow diverter 21 redirects flow upwardly 15, about an outside of the mouthpiece 11 being extended into the tube 20, and exiting laterally out a plurality of apertures 22 and back down again generally in a vortex-type circulation (FIG. 1) achieved by the geometry of the impact flow diverter 21 and shape and alignment of the apertures 22. Importantly, the flow characteristics attained as generally illustrated, provide the ability for the other unit components to work to ionize 13 and collect 23, as further detailed herein.

Also configured at the conclusion of the mouthpiece 11/flow diverter 21 and at an upper portion of the tube 20 is a negative electrode 13. In other embodiments the negative electrode 13 extends more downwardly into the tube 20. However, in this preferred FIG. 1 embodiment, the negative electrode 13 is not a typical rod or strip geometry, but instead a novel ring electrode 13, the ring electrode additionally contains metallic structure 14, or veins 14, at an interior of the ring while still allowing mostly free passage of air therethrough. The veins 14 are provided for increasing surface area of the negative electrode 13 in contact with the smoke or vapor accentuating propensity for stripping electrons while not overly hindering air flow in the device 10.

Additionally, with reference to FIG. 1 through FIG. 5, all components are mounted on an inner chassis 40 with an outer enclosure 30 covering and fitting over a top of the inner chassis 40. In other embodiments, the inner chassis 40 may itself be characterized as a replaceable exhale filter cartridge 40 that configures up and into an end of the outer enclosure 30. In its method of operation, a user exhales into the device 10, and air is sensed by the flow sensor switch 12 that provides power via battery 31 and step-up or high voltage generator (HVG) 32 which provides electrical potential to the system 10 negative electrode 13 and positive collector plate 23. A coiled collector plate 23 (in lieu of a planar collector plate) assists in providing a compact unit while increasing usable surface area. More particularly, the coiled collector plate 23 is a rolled copper sheet that allows air to pass therethrough.

Also, more specifically with regard to FIG. 1 and FIG. 5, exhale air flow velocity is slowed and diverted 21 to better allow for ionization of the smoke or vapor. More specifically, the impact flow diverter 21 piece provides circulation and vortexes the air allowing for efficient, rapid ionization, as shown in FIG. 1. With regard to FIG. 5 as well as FIG. 1, this circulation additionally allows for the collector plate 23 to successfully attract and attach the ionic smoke particles (illustrated in FIG. 5) thereto. An end cap (exit filter) 40 containing passive filter medium such as carbon, extruded carbon 41, HEPA 42 and/or mesh is subsequently provided for deodorizing and/or finish filter as well as removing any O₃ and other volatile compounds not neutralized by the ionization chamber 20. Importantly, the carbon filter is positioned before and upstream of the HEPA filter to increase absorption to the carbon filter and decrease clogging to the HEPA filter.

Yet still with regard to FIG. 1 through FIG. 5, the invention 10 is structurally an inner chassis 40 with all components mounted on (or inside) said chassis 40; further, with an outer enclosure 30 enclosing and housing all components, the outer enclosure 30 also includes an aperture 36 therethrough for mounting the mouthpiece 11. Since the high voltage generator (HVG) 32 requires a relatively large space in a compact device, the invention embodiment 10 provides that the high voltage generator 32 is mounted on the inner chassis 40 providing structural support thereto. The battery 31 is accordingly provided mounted adjacent to the HVG 32 on the inner chassis having a space saving profile configuration (FIG. 1). Terminals and connections 34, 35 on the high voltage generator 32 connect to the battery 31 and the negative electrode 13 with a first of the connections 34, via an air flow sensor switch triggered by the air flow sensor 12. The space saving profile includes a battery that is substantially wider and taller as compared to its thickness.

Specifically with regard to FIG. 4 and FIG. 5, the inner chassis has a hollowed-out interior for containing mechanical filter medium, for example, an extruded carbon filter 41 with channels therethrough (not shown) configured with or without an HEPA (High Efficiency Particulate Air) filter 42. This inner chassis 40 also serves as an end cap 40 to the device and could be separably removeable and replaceable. Additionally, the cylindrical tube 20, or broadly hollow chamber portion 20, is configured to an opening 46 in the inner chassis 40. It should be appreciated that the outer enclosure 30 is configured at least partially (if not wholly) over the inner chassis 40 and at least partially (if not wholly) encasing the hollow chamber portion 20. with regard to FIG. 1 and FIG. 5, the impact flow diverter piece 21 is needed for achieving the fluid mechanic properties for air flow about an inside of the tube 20 to allow ionization to take place. More particularly, air flow is redirected upwardly 15 and outwardly in a reverse direction from the initial exhale. Subsequently, air flow is directed to an outer portion of the device through a plurality of radially aligned apertures 22 and downwardly again. This circulating air movement creates a vortex of air ideal for passage through the negative electrode and allowing ionization to take place. The negative electrode is realized as a ring 13 at an entrance to the ionization tube with veins 14 extending inwardly providing more conducting surface area. Alternatively, the negative electrode could be realized as a strip suspending down into the tube. When the negative electrode 13 is energized via the high voltage generator (HVG) 32, an electric potential is created between electrodes, 13, 23 stripping electrons from particulate matter including ozone O₃ compounds. The ionic particulate matter is then attracted to the positive collector plate 23. As stated, in the preferred embodiment, the positive collector plate 23 is a copper sheet however rolled in a coil, thereby increasing surface area while remaining relatively compact.

Again, with regard to FIG. 1, the invention 10 requires a relatively high voltage and therefore a step-up high voltage generator (HVG) 32 is provided increasing terminal battery voltage to the negative electrode 13. The mouthpiece portion 11 comprises a one-way flow sensor 12 activating a one-way senor switch closing a circuit to energize the personal air filter 10 upon exhale; wherein an air flow is sensed by an air flow sensor and the one-way flow sensor switch closes an electrical circuit connecting 34 the battery 31 and the negative electrode 13.

Yet still further with regard to FIG. 1, FIG. 4, and FIG. 5, the inner chassis 40 is also an end cap coupled to an exit to the cylindrical tube 20. The inner chassis/end cap 40 further has a hollow interior containing mechanical, or passive, filter media; for example, extruded and baked carbon 41 having apertures allowing airflow therethrough, and a HEPA (High Efficiency Particulate Air) filter 42 medium. This arrangement is intended for filtering any smoke or harmful compounds not ionized or otherwise captured by the positive collector plate 23.

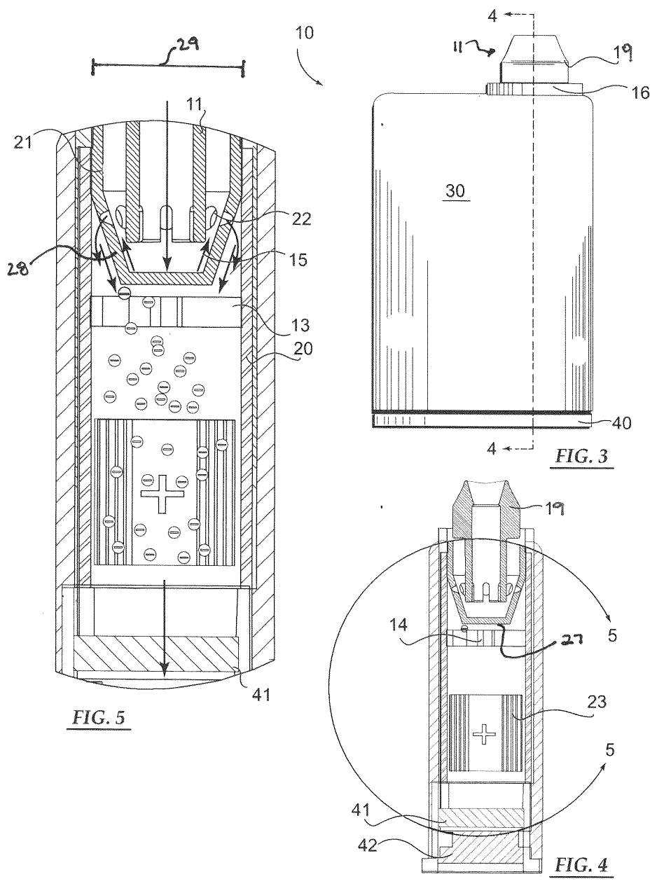

Regarding FIG. 3 a profile view is provided with all components assembled. As is apparent, the relatively wide profile is needed to accommodate the HVG 32. Ring 16 is optionally provided to seat the mouthpiece 11 and connect and seal same with the cylindrical tube 20.

Alternatively, the mouthpiece 11 could configure directly to the tube 20 and the outer enclosure 30. These components connect through apertures 36, 46 through the outer enclosure 30 and the end cap 40, respectively.

Additionally further regarding FIG. 4, the mouthpiece has a mushroom cap 19 being the portion of the mouthpiece directly above line 5-5 in FIG. 4. Extending beyond and beneath the mouthpiece mushroom cap 19, the mouthpiece includes a mouthpiece 11 cylindrical sidewall forming a hollow interior; this hollow interior being the initial axial flow shown with center arrow in FIG. 5. As compared to FIG. 1, FIG. 3, FIG. 4., and FIG. 5 more specifically show that mouthpiece 11 has a mushroom cap 19 and a hollow chamber portion, the cap 19 importantly directing airflow 15 back downward via apertures 22.

Further regarding the construction providing proper air flow in FIG. 5, (and in FIG. 4) the impact flow diverter piece 21 further comprises a bottom end wall 27, wherein it is shown by the arrows redirecting air flow upwardly. Configured to the impact flow diverter piece 21 is a slanted oblique sidewall 28 extending around the mouthpiece 11 cylindrical sidewall. Together with the bottom end wall 27, the oblique sidewall 28 redirects axial air flow of the mouthpiece (cylindrical sidewall forming the) hollow interior outwardly and upwardly, around an outside of the mouthpiece cylindrical sidewall forming the) hollow interior. Next, a plurality of apertures 22 are provided in the slanted oblique sidewall 28 (of the impact flow diverter piece 21), therethrough, wherein the mouthpiece mushroom cap 19 together with the plurality of apertures redirects air flow outwardly and downwardly into the tube for ionization, as shown.

It can be appreciated, with regard to an upper portion of FIG. 5, that the tube 20 includes a tube inner diameter 29 defining a circumference (the circumference(s) shown in FIG. 1), wherein the impact flow diverter piece 21 includes an impact flow diverter piece outer diameter defining a circumference, wherein the negative electrode 13 includes a negative electrode outer diameter defining a circumference, wherein the tube 20 inner diameter and the impact flow diverter piece 21 outer diameter and the negative electrode 13 outer diameter are substantially equal allowing for the impact flow diverter 21 and the negative electrode 13 to fit securely inside an upper portion of the tube 20 being a hollow exhale ionization chamber. The relationship is shown in FIG. 5 as reference character 29.

With regard to FIG. 1 and FIG. 4, it should be appreciated that the positive collector plate 23 could be realized as a copper coil or a metallic foil (possibly copper), the differences being thickness of each. For example, a foil would be approximately on the order of 0.2 mm thickness; and a sheet would be on the order of 0.5 mm to 1 mm in thickness, in the preferred embodiments. Also, the metallic sheet employed could be a mesh structure. The mesh essentially being a network of wire.

In an alternative embodiment, the high voltage generator 32 and/or battery 31 could sit outside the outer enclosure 30 in a pod (not illustrated) shaped and positioned specifically according to the size of these components. Particularly, with regard to the HVG 32, the system 10 would not function without this component because higher voltages are needed to strip electrons from smoke particulate. And further, the HVG 32 will take up the most space of any component housed within the device (FIG. 1 and FIG. 2). It will also be relatively heavy, so positioning of the HVG 32 will become a design consideration in light of maintaining a compact device 10.

While the particular Ionic Personal Air Filter herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

What is claimed is:

1. A personal air filter for filtering smoke or vapor exhale comprising:
a hollow chamber portion;
a mouthpiece in fluid communication with the hollow chamber portion for receiving user exhale introduced thereto;
an impact flow diverter piece for redirecting and circulating air flow configured in between the mouthpiece at an upper area hollow chamber portion
a negative electrode configured at the upper area of the hollow chamber portion downstream of the mouthpiece and the impact flow diverter piece, the negative electrode providing an electrical potential creating a plurality of ionized smoke or vapor particles; and
a positive collector plate at a lower portion of the hollow chamber portion for collecting the ionized smoke particles; and
a battery providing the electrical potential between the negative electrode and the positive electrode.

2. The personal air filter for filtering smoke or vapor exhale of claim 1 wherein the positive collector plate comprises a copper sheet rolled in a coil thereby increasing a surface area in contact with the ionized smoke or vapor inside the hollow chamber portion.

3. The personal air filter for filtering smoke or vapor exhale of claim 1 further comprising:
a step-up high voltage generator increasing the electrical potential between the negative electrode and the positive electrode;
a one-way flow sensor switch to energize the personal air filter upon exhale, wherein an air flow is sensed by an air flow sensor and the one-way flow sensor switch closes an electrical circuit connecting the battery and the negative electrode;
an end cap coupled to an exit to the hollow chamber portion comprising an extruded carbon filter, for filtering any smoke or vapor not ionized or otherwise captured by the positive collector plate; and
an outer enclosure containing the hollow chamber portion, the battery and the high voltage generator.

4. The personal air filter for filtering smoke or vapor exhale of claim 1, wherein the impact flow diverter piece further comprises apertures for subsequently redirecting the airflow outwardly and downwardly passing through the hollow chamber portion.

5. The personal air filter for filtering smoke or vapor exhale of claim 1, the negative electrode comprising:
a ring configured at an upper area of the hollow chamber portion; and
an additional metallic structure at an interior to the ring, the additional metallic structure providing added surface area stripping electrons from harmful compounds.

6. A personal air filter for filtering smoke or vapor exhale comprising:
a tube;
a mouthpiece in fluid communication with the tube for receiving user exhale introduced thereto;
an impact flow diverter piece for redirecting and circulating air flow configured in between the mouthpiece and the tube;
a negative electrode configured to the tube downstream of the mouthpiece and the impact flow diverter piece, the negative electrode providing electrical potential creating a plurality of ionized smoke particles; and
a positive collector plate at a lower portion of the tube for collecting the ionized smoke particles;
a battery delivering a voltage to the personal air filter and the negative electrode; and
a step-up high voltage generator for increasing the voltage to the negative electrode.

7. The personal air filter for filtering smoke or vapor exhale of claim 6 wherein the positive collector plate comprises a metallic mesh sheet rolled in a coil.

8. The personal air filter for filtering smoke or vapor exhale of claim 6 further comprising:
a one-way flow sensor senor switch to energize the personal air filter upon exhale, wherein an air flow is sensed by an air flow sensor and the one-way flow sensor switch closes an electrical circuit connecting the battery and the negative electrode;
an end cap coupled to an exit to the tube comprising an extruded carbon filter, for filtering any smoke not ionized or otherwise captured by the positive collector plate; and
an outer enclosure containing the tube, the battery and the high voltage generator.

9. The personal air filter of claim 6, wherein the mouthpiece includes a mouthpiece cylindrical sidewall forming a hollow interior, the mouthpiece cylindrical sidewall extending down into an interior of the impact flow diverter piece, the impact flow diverter piece further comprising:
a bottom end wall;
a slanted oblique sidewall, together with the bottom end wall, redirecting axial air flow of the mouthpiece hollow interior outwardly and upwardly, around an outside of the mouthpiece hollow interior, wherein the mouthpiece further includes a mouthpiece mushroom cap; and
a plurality of apertures in the slanted oblique sidewall, therethrough, wherein the mouthpiece mushroom cap together with the plurality of apertures redirects air flow outwardly and downwardly into the tube for ionization.

10. The personal air filter of claim 6, wherein the tube includes a tube inner diameter defining a circumference, wherein the impact flow diverter piece includes an impact flow diverter piece outer diameter defining a circumference, wherein the negative electrode includes a negative electrode outer diameter defining a circumference, wherein the tube inner diameter and the impact flow diverter piece outer diameter and the negative electrode outer diameter are substantially equal allowing for the impact flow diverter and the negative electrode to fit securely inside an upper portion of the tube being a hollow exhale ionization chamber.

11. The personal air filter of claim 6, the negative electrode comprising:
a ring configured at an upper portion the tube; and
an additional metallic structure at an interior to the ring, the additional metallic structure providing added surface area stripping electrons from harmful compounds.

12. A personal air filter for filtering smoke or vapor exhale comprising:
an inner chassis having an interior, the interior containing a filter medium;
a hollow chamber portion extending downwardly into an opening in the inner chassis;
a mouthpiece in fluid communication with the hollow chamber portion for receiving user exhale introduced thereto; and
an outer enclosure configured at least partially over the inner chassis and at least partially encasing the hollow chamber portion.

13. The personal air filter for filtering smoke or vapor exhale of claim 12,
an impact flow diverter piece for redirecting and circulating air flow configured at an exit of the mouthpiece and at an upper portion of the hollow chamber portion;
a negative electrode configured to the hollow chamber portion, the negative electrode providing electrical potential creating a plurality of ionized smoke or vapor particles; and
a positive collector plate at a lower portion of the hollow chamber portion for collecting the ionized smoke particles.

14. The personal air filter for filtering smoke or vapor exhale of claim 13 wherein the positive collector plate comprises a copper foil rolled in a coil thereby increasing surface area in contact with the ionized smoke particles.

15. The personal air filter of claim 13 further comprising:
a battery delivering a voltage to the personal air filter and the negative electrode;
a step-up high voltage generator increasing the voltage to the negative electrode; and
a one-way flow sensor senor switch to energize the personal air filter upon exhale; wherein an air flow is sensed by an air flow sensor and the one-way flow sensor switch closes an electrical circuit connecting the battery and the negative electrode.

15. The personal air filter for filtering smoke or vapor exhale of claim 12, the inner chassis further comprising an end cap portion coupled to an exit to the hollow chamber portion, wherein the filter medium comprises an extruded carbon filter, for filtering ionized smoke or vapor particles captured by the positive collector plate.

16. The personal air filter for filtering smoke or vapor exhale of claim 13 wherein the positive collector plate comprises a metallic foil rolled in a coil thereby increasing surface area in contact with the ionized smoke particles.

17. The personal air filter for filtering smoke or vapor exhale of claim 13 wherein the positive collector plate comprises a metalic mesh sheet rolled in a coil.
